SEQUENCE LISTING (4 sequences)

SEQ ID NO: 1 - protein, 34 aa, Mus musculus
DVSTSQAVLPDDFPRYPVGKFFQYDTWRQSAGRL

SEQ ID NO: 2 - protein, 34 aa, Rattus
DVSTSQAVLPDDFPRYPVGKFFKFDTWRQSAGRL

SEQ ID NO: 3 - protein, 34 aa, Homo sapiens
DVSTPPTVLPDNFPRYPVGKFFQYDTWKQSTQRL

SEQ ID NO: 4 - protein, 34 aa, Artificial Sequence
  Synthetically generated peptide
DVSTXXXVLPDXFPRYPVGKFFXXDTWXQSXXRL

(19) United States
(12) Patent Application Publication
(10) Pub. No.: US 2006/0116318 A1
(43) Pub. Date: Jun. 1, 2006

(54) Preptin methods of use

(76) Inventors: Jillian Cornish, Mission Bay (NZ); Ian Reginald Reid, Mount Albert (NZ); Garth James Smith Cooper, Ponsonby (NZ); Christina Maree Buchanan, Beach Haven (NZ)

Correspondence Address: FISH & RICHARDSON PC, P.O. BOX 1022, MINNEAPOLIS, MN, 55440-1022, US

(21) Appl. No.: 10/523,617

(22) PCT Filed: Jul. 31, 2003
(86) PCT No.: PCT/NZ03/00168

§ 371 Date: Jan. 6, 2006

Related U.S. Application Data
(60) Provisional application No. 60/400,443, filed on Aug. 1, 2002.

(51) Int. Cl. A61K 38/17

(52) U.S. Cl. 514/12

(57) ABSTRACT

This invention features a method for treating a bone condition in a patient, e.g., a mammal, a human, a horse, a dog, or a cat. The method includes administering an effective amount of preptin, preptin analog, or a preptin agonist to the patient.

### BACKGROUND

Preptin is a 34-amino acid peptide corresponding to Asp⁶⁹-Leu¹⁰² of the proinsulin-like growth factor II. It is present in pancreatic islet beta cells and undergoes glucose-mediated co-secretion with insulin.

The two-phase secretion of insulin in response to glucose is well characterized. See, e.g., Grodsky et al. (1968) Acta Diabetol. Lat. 5: 140-161. The first phase results in a transient spike in insulin secretion, while the second phase results in a progressive increase in insulin release. Preptin has been found to enhance, but not initiate, insulin secretion. More specifically, infusion of preptin into the isolated, perfused rat pancreas exerts a significant increase in the second phase of secretion, while removal of preptin from the pancreas by adding anti-preptin antibodies results in a decrease in both the first and the second phases of insulin secretion. See Buchanan et al. (2001) Biochem. J. 360: 431-439.

It also has been suggested that preptin elicits its effects by binding to a cell surface receptor (Buchanan supra).

### SUMMARY

The present invention is based, in part, on the discovery that preptin can stimulate proliferation of osteoblasts, which are known to play a role in mediating or modulating bone growth.

In one aspect, this invention features a method for treating a bone condition in a patient, e.g., a mammal, a human, a horse, a dog, or a cat. The method includes administering an effective amount of preptin, preptin analog, or a preptin agonist to the patient.

The patient can be suffering from a disease associated with excessive resorption or breakdown of bone tissue. Examples of such diseases include, but are not limited to, osteoporosis, osteopenia, bone defects, and osteogenesis imperfecta. The patient can also be suffering from bone loss as a result of immobility, bone fractures, malignancy, primary hyperparathyroidism, endocrine disorders, autoimmune arthritis, or addictive drug use. The patient can also be undergoing a treatment (e.g., corticosteroid treatment, bone marrow transplantation, or oophorectomy) known to result in bone loss. The term “bone condition” refers to any disease or symptom wherein mediation of osteoblast or osteoclast activity (or levels) is involved, and includes any of the diseases or situations described above.

As used herein, “preptin” is an isolated peptide of 34 amino acids in length, the sequence of which is described as in formula (I):
Asp Val Ser Thr R₁ R₂ R₃ Val Leu Pro Asp R₄ Phe Pro Arg Tyr Pro Val Gly Lys Phe Phe R₅ R₆ Asp Thr Trp R₇ Gln Ser R₈ R₉ Arg Leu formula (I)
Wherein:
R₁ is Ser or Pro
R₂ is Gln or Pro
R₃ is Ala or Thr
R₄ is Asp or Asn
R₅ is Gln or Lys
R₆ is Tyr or Phe
R₇ is Arg or Lys
R₈ is Ala or Thr, and
R₉ is Gly or Gln;
or an analog thereof.
   It includes mouse preptin, rat preptin, and human preptin, the sequences of which are shown below.
Mouse preptin: DVSTSQAVLPDDFPRYPVGKFFQYDTWRQSAGRL (SEQ ID NO: 1)
Rat preptin: DVSTSQAVLPDDFPRYPVGKFFKFDTWRQSAGRL (SEQ ID NO: 2)
Human preptin: DVSTPPTVLPDNFPRYPVGKFFQYDTWKQSTQRL (SEQ ID NO: 3).
   The amino acid sequence corresponds to Asp₆₉-Leu₁₀₂ of the proIGF-II E-peptide in each mammal.

Analogs of preptin include functional equivalents of preptin (e.g., functional equivalents of those of formula I). In terms of preptin itself, functional equivalents include all proteins which are immunologically cross-reactive with and have substantially the same function as preptin (e.g., any of SEQ ID NOs: 1-3). That equivalent may, for example, be a fragment of preptin containing from 6-33 amino acids (usually representing a C-terminal truncation) and including a preptin active site or sites, a substitution, addition or deletion mutant of preptin, or a fusion of preptin or a fragment or a mutant with other amino acids.

“A preptin agonist” is a compound which (1) has a high affinity (e.g., a Ki of 10⁻⁷-10⁻⁹ M, a Ki of 10⁻⁸-10⁻⁹ M) for a preptin-binding receptor (as defined by the receptor binding assay described in Motulsky, H. J and Mahan, L. C. (1984). Mol. Pharmacol. 25: 1; and (2) promotes the proliferation of bone cells, e.g., osteoblasts.

In one embodiment, the methods described herein include administering to a patient an effective amount of preptin having the amino acid sequence of SEQ ID NO: 1, 2, or 3.

In another embodiment, the method includes administering to a patient an effective amount of a preptin agonist having a fragment (e.g., any sequence between 17 and 33 amino acids in length, inclusive, of SEQ ID NO: 1, 2, or 3) or the entirety of the amino acid sequence of SEQ ID NO: 1, 2, or 3. For example, a preptin agonist is a peptide being 17-110 amino acids in length, e.g., a peptide having less than 87 amino acids, or more than 20 (e.g., any integer between 21 and 110, inclusive) amino acids, and containing, in consecutive sequence, any part of SEQ ID NO: 1, 2, or 3. In another example, a preptin agonist is a peptide having 35-110 amino acids in length, e.g., a peptide having less than 87 amino acids, or more than 35 (e.g., any integer between 35 and 110, inclusive) amino acids, and containing the entirety of the amino acid sequence of SEQ ID NO: 1, 2, or 3.

In a further embodiment, the method includes administering to a patient an effective amount of a preptin agonist containing an amino acid sequence that is at least 60% (e.g., 70%, 80%, 90%, 95%, or 98%) identical to SEQ ID NO: 1, 2, or 3. The “percent identity” of two amino acid sequences can be determined using the algorithm of Karlin and Altschul (1990, Proc. Natl. Acad. Sci. USA 87: 2264-2268), modified as in Karlin and Altschul (1993, Proc. Natl. Acad. Sci. USA 90: 5873-5877). Such an algorithm is incorporated into the NBLAST and XBLAST programs (version 2.0) of Altschul et al. (1990) J. Mol. Biol. 215: 403-10. BLAST protein searches can be performed with the XBLAST program, score=50, wordlength=3 to obtain amino acid sequences homologous to the peptide molecules described herein. Where gaps exist between two sequences, Gapped BLAST can be utilized as described in Altschul et al. (1997) Nucleic Acids Res. 25 (17): 3389-3402. When utilizing BLAST and Gapped BLAST programs, the default parameters of the respective programs (e.g., XBLAST and NBLAST) can be used.

In still another embodiment, the method includes administering to a patient an effective amount of a preptin agonist containing SEQ ID NO: 1, 2, or 3 with up to 14 (e.g., any integer between 1 and 14, inclusive) conservative amino acid substitutions. A “conservative amino acid substitution” is one in which an amino acid residue is replaced with another residue having a chemically similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Amino acid analogs (e.g., phosphorylated amino acids) are also contemplated in the present invention.

In another aspect, this invention features a method for increasing or maintaining bone density. The method includes administering to a subject (e.g., a mammal, a human, a horse, a dog, or a cat) in need thereof an effective amount of preptin, preptin analog, or a preptin agonist as described herein. As used herein, the subject may have a substantially normal bone density or the subject may be at risk of bone deterioration. Examples of these subjects include postmenopausal women, usually at age 50 and over, and men over 60 years of age.

In a further aspect, this invention features a method for stimulating osteoblast growth or modulating osteoblast apoptosis. The method includes administering to a subject in need thereof an effective amount of preptin, preptin analog, or a preptin agonist. The term “osteoblast” refers to bone-forming cells.

The method includes administering to the subject (including a subject identified as in need of such treatment, e.g., a subject in need of modulation of osteoblast activity) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

This invention also features an article of manufacture that includes a vessel containing preptin, preptin analog, or a preptin agonist; and instructions for use of preptin, preptin analog, or a preptin agonist for treatment of a bone condition by administering an effective amount of preptin, preptin analog, or a preptin agonist to a patient.

Also within the scope of this invention is an article of manufacture. The article includes packaging material; and contained within the packaging material, preptin, preptin analog, or a preptin agonist. The packaging material comprises a label that indicates that preptin, preptin analog, or a preptin agonist can be used for treating a bone condition (e.g., osteoporosis, osteopenia, bone defects, or osteogenesis imperfecta) in a patient. In other aspects, the label includes dosage information.

Other features, objects, and advantages of the invention will be apparent from the description and drawings, and from the claims.

### DESCRIPTION OF DRAWINGS

FIG. 1 depicts the effect of various concentrations of rat preptin or vehicle on cell number (FIG. 1A) and thymidine incorporation (FIG. 1B) in cultures of fetal rat osteoblasts. In this figure, * represents p<0.05, ** represents p<0.01, and *** represents p<0.001.

FIG. 2 depicts the effect of various concentrations of human preptin or vehicle on cell number (FIG. 1A) and thymidine incorporation (FIG. 1B) in cultures of fetal rat osteoblasts. In this figure, * represents p<0.05, ** represents p<0.01, and *** represents p<0.001.

FIG. 3 depicts the effects of two concentrations of rat preptin on thymidine incorporation in murine neonatal calavarial organ culture.

FIG. 4 depicts the effects of various concentrations of rat preptin on osteoclast development in vitro.

FIG. 5 depicts the effects of preptin on primary rat osteoblasts pre-treated with either MAP kinase inhibitors PD-98059 or U-0126 (FIG. 5A) or a Gᵢ protein inhibitor, pertussis toxin (FIG. 5B).

FIG. 6 depicts the effects of rat preptin on apoptosis of serum-deprived primary rat osteoblasts.

### DETAILED DESCRIPTION

This invention relates to use of preptin, preptin analog, or a preptin agonist for stimulating osteoblast growth or modulating osteoblast apoptosis. Preptin can be isolated from pancreatic islet beta cells in a manner as described in Buchanan et al. (2001) Biochem. J. 360: 431-439. Preptin, as well as a preptin analog or preptin agonist, also can be prepared by a synthetic method. More specifically, synthesis of peptides (e.g., preptin) is well established in the art. See, e.g., Stewart, et al. (1984) Solid Phase Peptide Synthesis (2^{nd} Ed.); and Chan (2000) “Fmoc Solid Phase Peptide Synthesis, A Practical Approach,” Oxford University Press. The peptides may be synthesized using an automated peptide synthesizer (e.g., a Pioneer™ Peptide Synthesizer, Applied Biosystems, Foster City, Calif.). For example, a peptide is prepared on methylbenzyhydrylamine resin followed by hydrogen fluoride deprotection and cleavage from the resin. The synthesized peptide can be further purified by a method such as affinity column chromatography or high pressure liquid chromatography. Standard physicochemical characterization techniques are known in the art, including NMR (¹³C, ¹H, ¹⁹F, or ³¹P) and IR, which can provide confirmatory evidence of the identity and purity of the synthetic products. Amino acid analysis can also be used to confirm the amino acid composition of the peptide. Mass spectroscopy can be used to identify the molecular weight of synthetic products.

One aspect of this invention is a method for treating a bone condition with an effective amount of a preptin, preptin analog, or a preptin agonist. Another aspect of this invention is a method for increasing or maintaining bone density with a preptin, preptin analog, or a preptin agonist. The term “treating” is defined as the application or administration of a composition including a preptin, preptin analog, or a preptin agonist to a patient, who has, or is determined to have, a bone condition, a symptom of a bone condition, a disease or disorder secondary to a bone condition, or a predisposition toward a bone condition, with the purpose to cure, alleviate, relieve, remedy, or ameliorate the bone condition, the symptom of the bone condition, the disease or disorder secondary to the bone condition, or the predisposition toward the bone condition. “An effective amount” refers to an amount of preptin, preptin analog, or a preptin agonist that confers a therapeutic effect on the treated subject. The therapeutic effect may be objective (i.e., measurable by some test or marker) or subjective (i.e., subject gives an indication of or feels an effect). An effective amount of preptin, preptin analog, or a preptin agonist described above may range from about 1 Tg/Kg body weight to about 1000 Tg/Kg body weight. Effective doses will also vary depending on the route of administration, as well as the possibility of co-usage with other agents for stimulating osteoblast growth or modulating osteoblast apoptosis, such as a bone anti-resorptive agent (e.g., calcitonin or bisphosphonate) or a bone anabolic agent (e.g., parathyroid hormone, parathyroid hormone related protein, cytokines, or growth hormone).

As used herein, preptin, preptin analog, and preptin agonists are defined to include pharmaceutically acceptable derivatives (e.g., salts).

Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate, trifluoroacetate, and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

Also within the scope of this invention is a pharmaceutical composition that contains an effective amount of preptin, preptin analog, or a preptin agonist, and a pharmaceutically acceptable carrier.

The term “pharmaceutically acceptable carrier” refers to a carrier (adjuvant or vehicle) that may be administered to a patient, together with preptin, preptin analog, or a preptin agonist, and which does not destroy the pharmacological activity thereof and is nontoxic when administered in doses sufficient to deliver preptin, preptin analog, or a preptin agonist.

Pharmaceutically acceptable carriers that may be used in the pharmaceutical compositions described above include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein. Oil solutions or suspensions may also contain a long-chain alcohol diluent or dispersant, or carboxymethyl cellulose or similar dispersing agents, which are commonly used in the formulation of pharmaceutically acceptable dosage forms such as emulsions and or suspensions.

To practice the method for treating a bone condition or the method for increasing or maintaining bone density, preptin, preptin analog, or a preptin agonist can be administered to a patient or a subject. The preptin, preptin analog, or the preptin agonist can, for example, be administered in a pharmaceutically acceptable carrier such as physiological saline, in combination with other drugs, and/or together with appropriate excipients. It also can, for example, be administered by injection, intravenously, intraarterially, subdermally, intraperitoneally, intramuscularly, or subcutaneously; or orally, buccally, nasally, transmucosally, topically, in an ophthalmic preparation, by inhalation, by intracranial injection or infusion techniques. The methods herein contemplate administration of an effective amount of compound or compound composition to achieve the desired or stated effect. Lower or higher doses than those described above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

A pharmaceutical composition may be orally administered in any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

A sterile injectable composition (e.g., aqueous or oleaginous suspension) can be formulated according to techniques known in the art using suitable dispersing or wetting agents (such as, for example, Tween 80) and suspending agents.

Topical administration of a pharmaceutical composition is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-applied transdermal patches are also included in this invention.

A pharmaceutical composition may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Preptin agonists can be tested for their abilities to stimulate osteoblast growth or modulate osteoblast apoptosis by examining their activities in the in vitro assays described herein. See the specific examples below. In vivo screening can also be performed by following procedures well known in the art. See, e.g., Cornish et al. (1997) Am J Physiol 273: E1113-E1120; and Cornish et al. (2000) Am J Physiol 279: E730-E735.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

The invention will be further described in the following examples. It should be understood that these examples are for illustrative purposes only and are not to be construed as limiting this invention in any manner.

### EXAMPLE 1

### Promoting Proliferation of Bone Cells

Osteoblast-Like Cell Culture. Osteoblasts were isolated from 20 day fetal rat calvariae as previously described (Cornish et al. (1999) American Journal of Physiology Endocrinology & Metabolism 277: E779-E783). Briefly, calvariae were excised and the frontal and parietal bones, free of suture and periosteal tissue, were collected. The calvariae were sequentially digested using collagenase and the cells from digests 3 and 4 were collected, pooled and washed. Cells were grown to confluence and then subcultured into 24 well plates. Cells were growth arrested in minimum essential medium (MEM)/0.1% bovine serum albumin for 24 h. Fresh media and experimental compounds were added for a further 24 h. Cells were pulsed with tritiated-thymidine two hours before the end of the experimental incubation. The effect of preptin on proliferation of fetal rat osteoblast-like cells was first assessed by the measurement of cell numbers. The effect of preptin on DNA synthesis in osteoblasts was then assessed by the measurement of [³H]-thymidine incorporation into isolated fetal rat osteoblast-like cells. There were 6 wells in each group and each experiment was repeated 3 or 4 times.

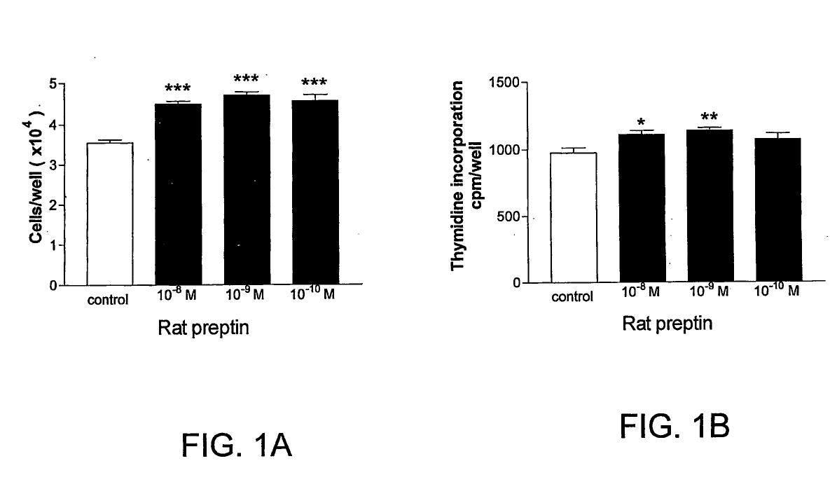

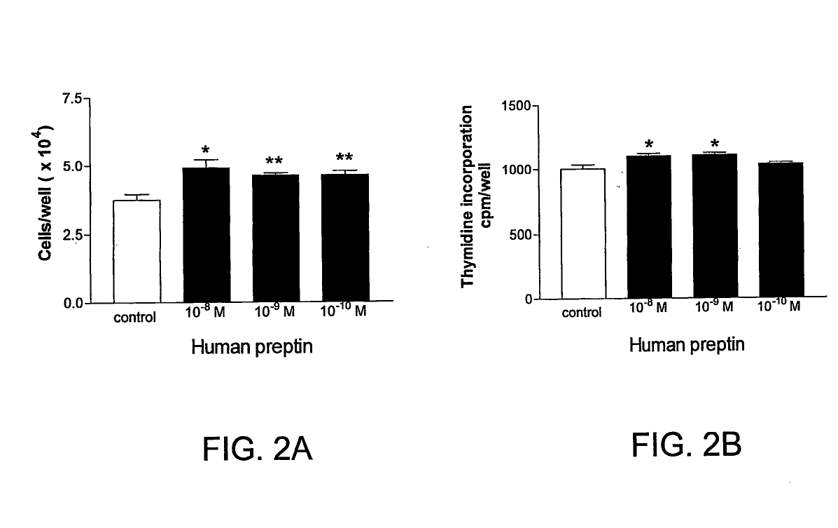

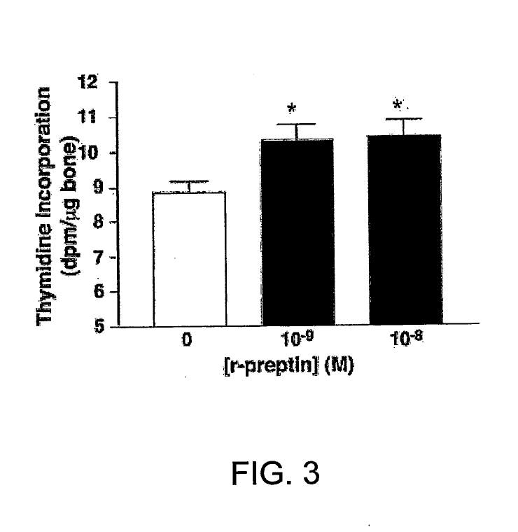

As shown in FIGS. 1 and 2, preptin, in a dose-dependent manner, stimulated the proliferation (cell number and DNA synthesis) of primary fetal rat osteoblasts and osteoblast-like cell lines at periphysiological concentrations (>10⁻¹¹M). In addition, thymidine incorporation was stimulated in murine neonatal calvarial organ culture (FIG. 3), likely reflecting the proliferation of cells from the osteoblast lineage.

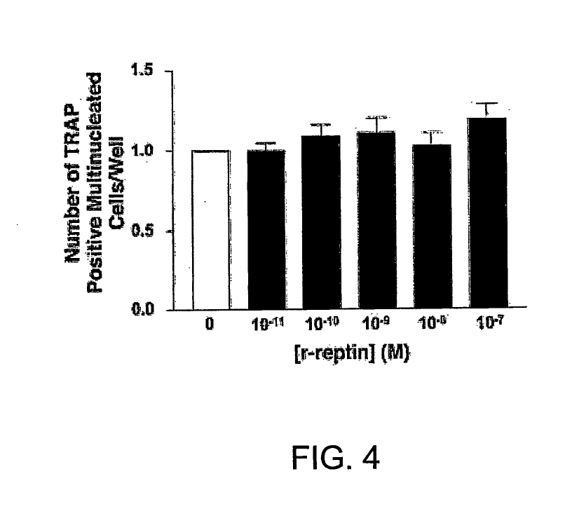

The effects of preptin on the development of osteoclasts was also examined. To assess osteoclast development, bone marrow is obtained from the long bones of normal mice, aged 4-6 weeks and cultured. Non-adherent cells are removed and the cultures are grown in the presence of 1α,25-dihydroxyvitamin D3 throughout the experiment. The cultures were maintained for 7 days and the number of tartrate-resistant acid phosphatase-positive multinucleated cells was assessed. An osteoclastogenesis assay was performed with murine bone marrow cultures in the presence of various concentrations of rat preptin. The number of tartrate-resistant acid phosphatase (TRAP)-positive multinucleated cells was assessed (FIG. 4). Preptin did not affect osteoclast development in this assay. Similarly, preptin did not affect bone resorption in a murine neonatal calavarial organ culture system (data not shown).

### EXAMPLE 2

### Inducing Phosphorylation of p42/p44 MAP Kinases In Bone Cells

Immunoblotting (Cell Signalling Methods). Primary rat osteoblasts prepared as described above were seeded in 6-well tissue culture plates at an initial density of 5×10⁴ cells/mL in MEM 5% FCS, and grown to 80-90% confluence. After serum starvation overnight, cells were treated at room temperature with test substances in MEM 0.1% BSA. In experiments designed to determine the effect of inhibitors of signal transduction on preptin-induced p42/44 MAP kinase phosphorylation, the cells were pre-treated with the inhibitor for 30 min prior to addition of test substances. The exception was pertussis toxin, which was added 18 h prior to test substances. After treatment for the indicated period of time, the treatment medium was aspirated, the cells were washed in ice-cold PBS and then scraped in ice-cold HNTG lysis buffer (50 mM HEPES, pH 7.5, 150 mM NaCl, 1% Triton, 10% glycerol, 1.5 mM MgCl₂, 1 mM EDTA) containing a cocktail of protease and phosphatase inhibitors (1 mM PMSF, 1 μg/mL peptatin, 10 μg/mL leupeptin, 10 μg/mL aprotinin, 1 mM sodium vanadate, 500 mM NaF). The lysates were briefly vortexed, clarified by centrifugation at 13,000 rpm at 4° C., then stored at −70° C. until analyzed. Protein content of the cell lysates was measured using the DC protein assay (BioRad, Hercules, Calif.). Equal amounts of whole cell lysate (30-50 μg) were subjected to 8% SDS-PAGE, transferred to nitrocellulose membranes and immunoblotted overnight at 4° C. with an anti-phospho-p42/44 MAP kinase antibody (1:1000). As a control for protein loading, the same filters were stripped and re-probed with an antibody to total p42/44 MAP kinase (1:400). Incubation with the HRP-conjugated secondary antibody was for 1 h at room temperature, and the membranes were analyzed by ECL. TUNEL assay (Apoptosis Method). Apoptosis in cultures of primary rat osteoblasts was assessed using the TUNEL method (DeadEnd™, Promega, Madison, Wis.) according to the manufacturer's instructions. Cells were seeded in 8-well chamber slides at 5×10⁴ cells/mL in MEM containing 5% FCS. Twenty-four hours later, the medium was changed to MEM/0.1% BSA and the cells incubated overnight. The following morning, test substances were added in fresh MEM/0.1% BSA for 18 h. At the end of the treatment period, cells were fixed in 2% paraformaldehyde for 15 min, then permeabilized with 1% Triton in PBS for 5 min. Thereafter, biotinylated nucleotides were added in the presence of terminal deoxynucleotidyl transferase for 1 h at 37° C. and the reaction terminated with 2×SSC. Endogenous peroxidases were blocked with 0.3% H₂O₂ for 5 min, streptavidin-HRP added for 30 min, and apoptotic nuclei colorized by addition of diaminobenzidine/H₂O₂ mixture. After counterstaining with hematoxylin, the number of apoptotic nuclei per microscopic field was counted and expressed as a proportion of that observed in the cells exposed to serum starvation throughout the entire treatment period. Each experiment was performed at lest twice and involved assessment of at least 6 chambers per treatment condition.

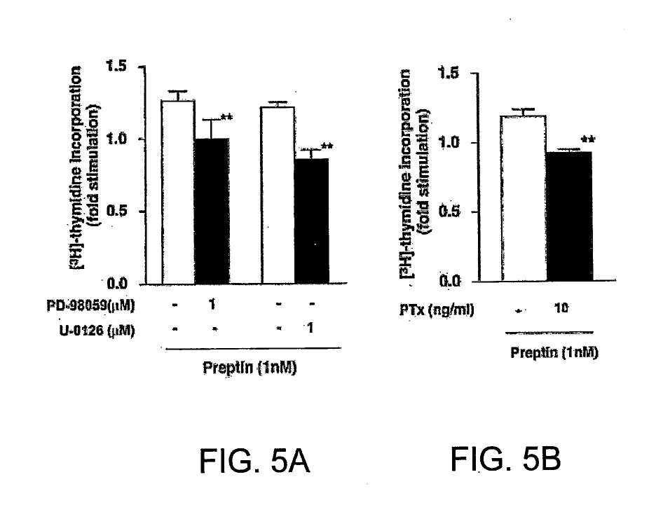

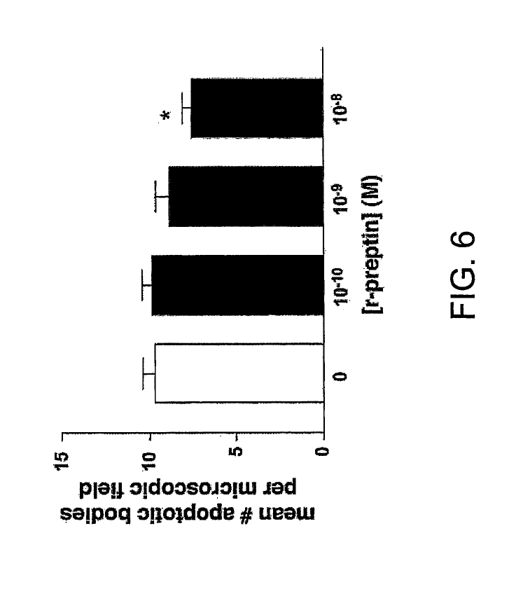

Preptin induced phosphorylation of p42/p44 MAP kinases in osteoblastic cells in a dose-dependent manner (₁₀⁻⁸-10⁻¹⁰M), as assessed by immunoblotting. The proliferative effects of preptin on primary osteoblasts were blocked when the cells were pre-treated with either of the MAP kinase kinase inhibitors PD-98059 or U-0126 (FIG. 5A), or a Gᵢ protein inhibitor, pertussis toxin (FIG. 5B). The effect of preptin on primary osteoblast apoptosis induced by serum deprivation was assessed. Apoptotic cells were detected by light microscopy using a modified TUNEL assay. Preptin had anti-apoptotic effects, at 10⁻⁸ M with treatment/control ratio of 0.78±0.08 (FIG. 6).

### EXAMPLE 3

### Promoting Bone Growth In Vivo

Several hormones that regulate nutritional status also impact on bone metabolism. Preptin, a 34-amino acid peptide hormone that increases glucose-mediated insulin secretion, has been recently isolated from the same secretory vesicles that contain insulin and amylin from the pancreatic β-cells. Preptin is anabolic to osteoblasts but, unlike amylin, does not regulate osteoclast activity.

Preptin not only stimulates osteoblast proliferation but also osteoblast differentiation at 10⁻⁸M, significantly increasing the number of mineralized bone nodules in long-term osteoblast cultures. These effects are also seen in vivo, when preptin is injected locally over the hemicalvariae of sexually mature male mice. After five daily subcutaneous injections of 16.5 micrograms of preptin, there was a significant increase in bone area, and mineralizing surface.

Methods

Three groups of sexually mature male mice were given twice daily subcutaneous injections over the right hemicalvaria for 5 consecutive days. Two groups received one of 2 doses of preptin (0.825 μg or 8.25 μg per injection), and a further two group received vehicle. The animals were sacrificed 1 week following the last injection. Fluorochrome labels were injected subcutaneously at the base of the tail on days 1 (calcein), 5 (alizarin red) and 14 (calcein) to measure dynamic histomorphometric indices (such as, extent of mineralizing surface). Calvariae were excised, fixed in 10% neutral-buffered formalin, dehydrated and embedded in methylmethacrylate resin. Sections were cut, mounted on gelatin-coated slides, and histomorphometric indices measured using image analysis.

Results

Local injections of high dose preptin significantly (by Student's t test) increased the bone area as well as the mineralizing surface compared to the control (see tables below).

TABLE 1
The effects of preptin on bone area in vivo
Bone Area

| | Control | Preptin (high) | Preptin (low) |
|---|---|---|---|
| Mean ± Std Error | 1.028 ± 0.023 | *1.084 ± 0.017 | 1.083 ± 0.027 |

*P = 0.013

TABLE 2
The effects of preptin on mineralizing surface of bone in vivo
% Mineralizing Surface/Total Bone Surface

| | Control | Preptin (high) | Preptin (low) |
|---|---|---|---|
| Mean ± Std Error | 1.048 ± 0.013 | *1.119 ± 0.032 | 1.065 ± 0.033 |

*P = 0.046

In conclusion, preptin, a peptide contained within proIGF-II, is anabolic to bone in in vitro and in vivo models. Since it is secreted from the pancreatic β-cell, it may act in concert with the other β-cell hormones, insulin and amylin, to stimulate bone formation in hyperinsulinemic states, such as obesity. Preptin may also contribute to the osteosclerotic phenotype observed in patients with chronic hepatitis C infection who have increased circulating levels of proIGF-II, which contains the preptin peptide (Khosla S, Ballard F J, Conover C A. (2002) J Clin Endocrinol Metab. 87 (8):3867-70). Thus, the anabolic effects of preptin seen in rodent models may influence bone density in humans.

### OTHER EMBODIMENTS

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a bone condition, comprising administering to a patient in need thereof an effective amount of preptin, preptin analog, or a preptin agonist.

2. The method of claim 1, wherein the amino acid sequence of preptin is SEQ ID NO: 1, 2, or 3.

3. The method of claim 1, wherein the preptin agonist comprises a fragment or the entirety of the amino acid sequence of SEQ ID NO: 1, 2, or 3.

4. The method of claim 3, wherein the fragment is amino acid residues 17-34 of SEQ ID NO: 1, 2, or 3.

5. The method of claim 1, wherein the preptin agonist comprises an amino acid sequence that is at least 60% identical to SEQ ID NO: 1, 2, or 3.

6. The method of claim 5, wherein the preptin agonist comprises an amino acid sequence that is at least 80% identical to SEQ ID NO: 1, 2, or 3.

7. The method of claim 5, wherein the preptin agonist comprises an amino acid sequence that is at least 90% identical to SEQ ID NO: 1, 2, or 3.

8. The method of claim 5, wherein the preptin agonist comprises an amino acid sequence that is at least 95% identical to SEQ ID NO: 1, 2, or 3.

9. The method of claim 1, wherein the preptin agonist comprises SEQ ID NO: 1, 2, or 3 with up to 14 conservative amino acid substitutions.

10. The method of claim 9, wherein the preptin agonist comprises SEQ ID NO: 1, 2, or 3 with up to 10 conservative amino acid substitutions.

11. The method of claim 9, wherein the preptin agonist comprises SEQ ID NO: 1, 2, or 3 with up to 6 conservative amino acid substitutions.

12. The method of claim 9, wherein the preptin agonist comprises SEQ ID NO: 1, 2, or 3 with up to 2 conservative amino acid substitutions.

13. A method for increasing or maintaining bone density, comprising administering to a subject in need thereof an effective amount of preptin, preptin analog, or a preptin agonist.

14. The method of claim 13, wherein the amino acid sequence of preptin is SEQ ID NO: 1, 2, or 3.

15. The method of claim 13, wherein the preptin agonist comprises a fragment or the entirety of the amino acid sequence of SEQ ID NO: 1, 2, or 3.

16. The method of claim 15, wherein the fragment is amino acid residues 17-34 of SEQ ID NO: 1, 2, or 3.

17. The method of claim 13, wherein the preptin agonist comprises an amino acid sequence that is at least 60% identical to SEQ ID NO: 1, 2, or 3.

18. The method of claim 17, wherein the preptin agonist comprises an amino acid sequence that is at least 80% identical to SEQ ID NO: 1, 2, or 3.

19. The method of claim 17, wherein the preptin agonist comprises an amino acid sequence that is at least 90% identical to SEQ ID NO: 1, 2, or 3.

20. The method of claim 17, wherein the preptin agonist comprises an amino acid sequence that is at least 95% identical to SEQ ID NO: 1, 2, or 3.

21. The method of claim 13, wherein the preptin agonist comprises SEQ ID NO: 1, 2, or 3 with up to 14 conservative amino acid substitutions.

22. The method of claim 21, wherein the preptin agonist comprises SEQ ID NO: 1, 2, or 3 with up to 10 conservative amino acid substitutions.

23. The method of claim 21, wherein the preptin agonist comprises SEQ ID NO: 1, 2, or 3 with up to 6 conservative amino acid substitutions.

24. The method of claim 21, wherein the preptin agonist comprises SEQ ID NO: 1, 2, or 3 with up to 2 conservative amino acid substitutions.

25. A method for stimulating osteoblast growth or modulating osteoblast apoptosis, comprising administering to a subject in need thereof an effective amount of preptin, preptin analog, or a preptin agonist.

26. The method of claim 25, wherein the amino acid sequence of preptin is SEQ ID NO: 1, 2, or 3.

27. The method of claim 25, wherein the preptin agonist comprises a fragment or the entirety of the amino acid sequence of SEQ ID NO: 1, 2, or 3.

28. The method of claim 27, wherein the fragment is amino acid residues 17-34 of SEQ ID NO: 1, 2, or 3.

29. The method of claim 25, wherein the preptin agonist comprises an amino acid sequence that is at least 60% identical to SEQ ID NO: 1, 2, or 3.

30. The method of claim 29, wherein the preptin agonist comprises an amino acid sequence that is at least 80% identical to SEQ ID NO: 1, 2, or 3.

31. The method of claim 29, wherein the preptin agonist comprises an amino acid sequence that is at least 90% identical to SEQ ID NO: 1, 2, or 3.

32. The method of claim 29, wherein the preptin agonist comprises an amino acid sequence that is at least 95% identical to SEQ ID NO: 1, 2, or 3

33. The method of claim 19, wherein the preptin agonist comprises SEQ ID NO: 1, 2, or 3 with up to 14 conservative amino acid substitutions.

34. The method of claim 33, wherein the preptin agonist comprises SEQ ID NO: 1, 2, or 3 with up to 10 conservative amino acid substitutions.

35. The method of claim 33, wherein the preptin agonist comprises SEQ ID NO: 1, 2, or 3 with up to 6 conservative amino acid substitutions.

36. The method of claim 33, wherein the preptin agonist comprises SEQ ID NO: 1, 2, or 3 with up to 2 conservative amino acid substitutions.

37. An article of manufacture comprising:
a vessel containing preptin, preptin analog, or a preptin agonist; and
instructions for use of preptin, preptin analog, or a preptin agonist for treatment of a bone condition comprising administering an effective amount of preptin, preptin analog, or a preptin agonist to a patient.

38. An article of manufacture comprising:
packaging material; and
contained within the packaging material, preptin, preptin analog, or a preptin agonist;
wherein the packaging material comprises a label that indicates that preptin, preptin analog, or a preptin agonist can be used for treating a bone condition in a patient.

39-52. (canceled)
